(19) United States
(12) Patent Application Publication
(10) Pub. No.: US 2023/0350177 A1
(43) Pub. Date: Nov. 2, 2023

(54) ROBOTIC MICROSCOPE AND CONTROL THEREOF

(71) Applicant: Brainlab AG, Munich (DE)

(72) Inventors: Stefan VILSMEIER, Munich (DE); Sven FLOSSMANN, Munich (DE)

(21) Appl. No.: 17/791,275

(22) PCT Filed: Jan. 8, 2020
(86) PCT No.: PCT/EP2020/050323

§ 371 Date: Jul. 7, 2022

(51) Int. Cl. G02B 21/00; G05D 3/20

(52) U.S. Cl. CPC G02B 21/0012; G05D 3/20; G06F 3/012

(57) ABSTRACT

Provided is a robotic microscope that includes an optical system being moveable in one or more spatial directions and an actuator configured to move the optical system in the spatial directions. The microscope includes a capacitive sensor with an electrode coupled to at least a part of the optical system, the capacitive sensor configured to provide a sensor signal indicative of a capacitance in a vicinity of the electrode. The microscope further includes control circuitry configured to determine, based on processing the sensor signal, at least one capacitance value for the capacitance, and configured to actuate, based on comparing at least one capacitance value with at least one reference value for the capacitance, the actuator to move the optical system such that a distance between a user's head and part of the optical system and/or an orientation of the user's head and part of the optical system is substantially constant.

### FIELD OF THE INVENTION

The present invention generally relates to the field of robotic microscopes. The invention further relates to the use of a robotic microscope for medical and/or surgical procedures and to a method of operating a robotic microscope.

### TECHNICAL BACKGROUND

In many technical fields, including medical applications or procedures, such as surgical procedures, robotic microscopes are utilized. Usually, robotic microscopes comprise an optical system that can be controlled, displaced and/or moved in multiple degrees of freedom. For instance, robotic microscopes and/or the optical systems thereof can be moved, displaced and/or positioned by means of one or more actuators in one or more spatial directions relative to a site of interest, such as e.g. a surgical site on a patient, in order to provide a preferably unobscured view on the site of interest along a preferred viewing direction for the user or surgeon using the microscope. Apart from a positional control of the microscope and/or the optical system thereof, also a speed of the movement of the microscope and/or the optical system can be controlled by a user or surgeon for many robotic microscopes.

In order to actuate the one or more actuators of the microscope and hence to provide a positional control and/or a speed control, robotic microscopes are usually equipped with one or more switches, e.g. manually actuatable switches and/or hand switches, one or more joysticks, one or more foot pedals or the like, which are operatively coupled with the one or more actuators. Also, robotic microscopes can employ mouth pieces that can be actuated by a user or surgeon in order to position the microscope at a desired position and/or with a desired speed of movement. Apart from that, one or more markers, e.g. located on a forehead of the user or surgeon, may be tracked by a video camera in order to actuate the one or more actuators and to control the robotic microscope. Therein, control elements, such as switches, foot pedals and mouth pieces may be inconvenient and uncomfortable to use for the operator. The same applies to markers attached to the user's skin, wherein also additional effort may be required for applying the markers on the user's skin and/or on a cap, and for removing them therefrom.

It may, therefore, be desirable to provide an improved robotic microscope for arbitrary applications, such as e.g. medical procedures and/or surgical procedures, in which the above-mentioned drawbacks are at least mitigated or at least partly overcome.

The present invention can be used for any procedure involving a robotic microscope, e.g. a standalone microscope and/or in connection with another device or apparatus. In particular, the present invention can be used for medical procedures, e.g. in connection with surgical products like cranial navigation and/or spine navigation.

Aspects of the present disclosure, examples and exemplary steps or features and their embodiments are disclosed in the following. Different aspects, embodiments, examples and exemplary features of the present disclosure can be combined in accordance with the disclosure or invention wherever technically expedient and feasible.

### GENERAL DESCRIPTION OF THE INVENTION

In this section, a description of the general features and/or steps of the present invention or disclosure is given for example by referring to possible embodiments of the invention or disclosure. The invention, however, is defined in the independent claims, wherein further embodiments are incorporated in the dependent claims as well as the foregoing and following description.

As stated above, it may be desirable to provide for an improved robotic microscope, in which the above-mentioned drawbacks of conventional or currently used robotic microscopes are at least mitigated or at least partly overcome.

This is achieved by the subject matter of the independent claims, wherein further embodiments are incorporated in the dependent claims and the following description.

According to a first aspect of the disclosure, there is provided a robotic microscope. The robotic microscope (also referred to as “microscope” in the following) comprises an optical system, the optical system being movable in one or more spatial directions. Further, the robotic microscope comprises at least one actuator configured to move the optical system in and/or along the one or more spatial directions. The robotic microscope further comprises at least one capacitive sensor (also referred to as “capacitance sensor” or “sensor” hereinafter) with at least one electrode coupled to, arranged at, and/or mounted to at least a part of the optical system, wherein the capacitive sensor is configured to provide, generate and/or output one or more sensor signals indicative of a capacitance in a vicinity of the at least one electrode. Moreover, the robotic microscope comprises a control circuitry configured to determine, based on processing the one or more sensor signals of the capacitive sensor, at least one capacitance value for the capacitance in the vicinity of the at least one electrode, wherein the control circuitry is further configured to actuate and/or control, based on comparing the at least one capacitance value with at least one reference value for the capacitance (also referred to as “reference capacitance value” hereinafter), the at least one actuator to move the optical system, such that a distance between a head of a user of the microscope and the at least a part of the optical system and/or an orientation of the head of the user and the at least part of the optical system is substantially constant, substantially kept constant and/or is constant.

Accordingly, a user, operator and/or surgeon operating and/or using the microscope can position, control a position and/or control a movement of the microscope's optical system, and hence a field of view and/or a viewing angle of the microscope, e.g. onto a site of interest and/or a surgical site, in a touchless manner by moving its head. Therein, “touchless” may mean that the user may not necessarily have to touch, be in contact with and/or manually actuate a control element for controlling a position and/or a speed of a movement of the optical system, such as e.g. a switch, a hand switch, a joystick and/or a foot pedal. The touchless control of the microscope's optical system may also be of particular advantage in terms of a hygiene and additional efforts and costs, e.g. associated with a cleaning procedure, may be avoided, when compared to manually actuatable or positionable microscopes. Apart from that, also markers, which may be used in some conventional robotic microscopes to control the position of the optical system based on tracking the markers with a video camera, may not be required. This in turn, may significantly increase a comfort and convenience for the user and may safe additional effort associated with e.g. a procedure of applying markers to the user or removing markers from the user. In addition, positioning, controlling the position and/or controlling the movement of the optical system based on and/or by moving the head may provide an intuitive way for the user to control the microscope and/or the optical system thereof in a reliable, precise and/or accurate manner.

The robotic microscope may be in particular suitable for and/or may be configured for medical applications and/or procedures, such as e.g. surgical procedures. Accordingly, the robotic microscope may refer to a robotic surgical microscope. It should be noted, however, that the robotic microscope according to the present disclosure can also be used to advantage in many other technical fields and is therefore not limited to medical applications. Accordingly, the robotic microscope may be an optical microscope or any other type of microscope. Further, it should be noted that the term “robotic microscope” may refer to a microscope having an optical system that is at least partly actuatable, positionable, movable and/or controllable by one or more actuators. Moreover, the robotic microscope may refer to an automated and/or at least partly automated microscope.

The optical system of the microscope may, for instance, comprise an optics, e.g. including one or more lenses, one or more mirrors, one or more optical sensors or the like. The optics of the optical system may be positioned, arranged, and/or located relative to a site of interest, which is to be examined by means of the microscope, such as e.g. a surgical site. Therein, the optical system and/or the optics may be positioned and/or arranged relative to the site of interest at a desired position and/or at a desired viewing angle with respect to and/or relative to the site of interest. The optical system may further comprise an eyepiece, via which the user may view, see and/or examine the site of interest, e.g. directly based on light emitted or reflected at the site of interest or indirectly based on a display device arranged in the eyepiece.

Further, the optical system or at least a part thereof may be movable and/or displaceable in one or more spatial directions. In particular, the optical system or at least a part thereof may be moved and/or displaced by the at least one actuator e.g. in two or three orthogonal spatial directions. Therein, each of the spatial directions may define an axis of the microscope, along which the optical system can be moved and/or displaced, and/or around which the optical system can be rotated. Moreover, also a speed and/or velocity, with which the optical system and/or at least a part thereof can be moved in the one or more spatial directions and/or rotated around the one or more axes may be adjusted and/or controlled by the at least one actuator and/or the control circuitry. Accordingly, the robotic microscope and/or the optical system may have one or more degrees of freedom. For instance, the optical system may have three degrees of freedom for positioning the optical system in three-dimensional space along three orthogonal spatial directions and three degrees of freedom for rotating the optical system around three axes, e.g. as defined by the three spatial directions. Also, the speed and/or velocity, with which the optical system can be moved and/or displaced in the three spatial directions and/or rotated around the three axes can be adjusted and/or controlled.

Further, the at least one actuator may be any type or kind of actuator. For instance, the at least one actuator may refer to and/or comprise one or more electric motors, one or more hydraulic actuators, one or more pneumatic actuators or any other type of actuator.

In the context of the present disclosure, the at least part of the optical system, to which the at least one electrode is coupled, may refer to a part of the optical system that is movable and/or configured to move in accordance with a movement of the head of the user, e.g. in order to position and/or orient the optical system relative to the site of interest as desired by the user. For instance, the at least part of the optical system may refer to and/or may comprise the eyepiece of the optical system, a support structure of the optical system, a housing of the optical system, a frame of the optical system and/or any other part or member of the optical system.

The at least one capacitive sensor and/or the at least one electrode of the capacitive sensor may be mechanically coupled to the at least part of the optical system. For instance, the capacitive sensor and/or the at least one electrode may be coupled to the at least part of the optical system, such that the capacitive sensor and/or the at least one electrode moves in accordance with a movement, a motion and/or a displacement of the at least part of the optical system. Further, it should be noted that the capacitive sensor may comprise one or more electrodes. In particular, the capacitive sensor may comprise a plurality of electrodes, an arrangement of electrodes and/or an array of electrodes.

In the context of the present disclosure, the control circuitry may refer to a control circuit, a controller and/or a control unit. The control circuitry may comprise one or more processors. Further, the control circuitry may be operatively and/or electrically coupled to the capacitive sensor. Further, the control circuitry may be operatively and/or electrically coupled to the at least one actuator. The control circuitry may, for instance, be configured to process the sensor signal and derive the capacitance value from the sensor signal. Further, the control circuitry may be configured to determine a control signal based on comparing the determined capacitance value with the reference value. Based on the control signal, the at least one actuator may be actuated, controlled and/or instructed to move and/or displace the optical system. Further, the control circuitry may be configured to monitor the capacitance and/or the capacitance value of the capacitance in the vicinity of the at least one electrode. Therein, monitoring may refer to determining over time.

Therein, the control circuitry may be configured to actuate and/or control the at least one actuator to move the optical system, such that the distance between the user's head and the at least part of the optical system is substantially constant, substantially kept constant and/or constant. The distance may be a relative or an absolute distance between the head and the electrode. Further, the control circuitry may be configured to actuate the at least one actuator such that the relative orientation of the user's head and the at least part of the optical system is substantially constant, substantially kept constant and/or is constant. This may mean that a relative orientation and/or a relative angular position of the user's head with respect to the at least part of the optical system along and/or around at least one axis is kept substantially constant. Therein, the at least one axis may be defined by the at least one spatial direction, along which the optical system can be moved. Further, the term “substantially constant” may mean in the context of the present disclosure that the distance and/or orientation is kept constant to about 80%, preferably to about 90%, even more preferably to about 95%.

In the context of the present disclosure, the reference value for the capacitance may refer to a reference capacitance value indicative, representative and/or descriptive of the capacitance value of the at least one electrode with the user's head being positioned at a reference position and/or in a reference orientation of the user's head relative to the at least one electrode and/or relative to the optical system. Accordingly, the reference capacitance value may refer to the capacitance value that is expected, when the user's head is at the reference position and/or in the reference orientation relative to the at least one electrode and/or relative to the optical system.

Further, the sensor signal and/or the capacitance value may be indicative, descriptive and/or representative of at least one of a capacitance between the at least one electrode and a ground (or ground potential), between the at least one electrode and an outer surface of the user's head, between the at least one electrode and a reference electrode of the capacitance sensor, and between two electrodes of the capacitance sensor, e.g. if the capacitive sensor comprises a plurality of electrodes. Alternatively or additionally, the capacitance in the vicinity of the at least one electrode may be at least one of a capacitance between said at least one electrode and a ground (or ground potential), between said at least one electrode and an outer surface of the head, between said at least one electrode and a reference electrode of the capacitance sensor, and between said at least one electrode and a further electrode of the capacitance sensor, e.g. if the capacitive sensor comprises a plurality of electrodes.

The capacitance value of the at least one electrode may be determined, e.g. by the control circuitry, based on applying a known charge to said electrode and measuring a potential, by applying a known potential to said electrode and measuring a charge of the electrode (e.g. based on integrating the current supplied to the electrode over time), by constructing an oscillating circuit comprising said electrode, whereby the frequency or resonance frequency of that circuit may depend on the capacitance value, and measuring the frequency or resonance frequency of the oscillating circuit. In another example implementation, a capacitance bridge may be employed to measure the capacitance value of said electrode.

As described above, the at least part of the optical system, to which the at least one electrode is coupled, may, for instance, refer to and/or comprise the eyepiece of the optical system. During operation of the microscope, the user's head may be arranged adjacent to, close to and/or in the vicinity of the at least part of the optical system, e.g. the eyepiece. Therein, the head of the user may be spaced apart from the electrode and/or may not touch the electrode. It should be noted, however, that the at least one electrode may be covered by an electrically insulating cover and/or layer, and the user's head may be in contact with the cover and/or layer covering the electrode. If the user moves its head, an electrical permittivity of the material in the vicinity of the at least one electrode may be changed, thereby changing the capacitance in the vicinity of the at least one electrode. Such change in the capacitance can then be determined, monitored, sensed and/or detected by the control circuitry based on processing the sensor signal and/or based on determining the capacitance value. Further, the control circuitry can compare the determined capacitance value with the reference value for the capacitance, and move, displace and/or actuate the at least one actuator to move the optical system, such that the determined capacitance value substantially matches and/or equals the reference value for the capacitance. Accordingly, this may allow to precisely move, position and/or displace the optical system of the microscope in a reliable, efficient, convenient and precise manner, in particular in a touchless manner and/or without manual actuation of e.g. a switch, a joystick, a foot pedal, a mouth piece or the like.

According to an embodiment, the control circuitry is configured to determine, compute and/or calculate a deviation of and/or a difference between the at least one capacitance value and the at least one reference value, wherein the control circuitry is configured to actuate the at least one actuator to move the optical system, such that the deviation of and/or the difference between the at least one capacitance value and the at least one reference value is minimized, thereby keeping the distance between the head of the user and the at least part of the optical system and/or the orientation of the head of the user and the at least part of the optical system substantially constant and/or constant. By way of example, the control circuitry may compute the difference and/or deviation of the capacitance value and the reference value and generate a control signal corresponding to the determined difference and/or deviation. The control signal may then be provided to the at least one actuator to move the optical system, such that the deviation and/or difference is minimized and/or such that the determined capacitance value substantially matches and/or quals the reference value of the capacitance.

According to an embodiment, the at least one capacitance value is indicative, representative and/or descriptive of a capacitance between the at least one electrode and the head of the user. Alternatively or additionally, the at least one capacitance value is indicative, representative and/or descriptive of a capacitance between the at least one electrode and ground (or ground potential).

According to an embodiment, the capacitive sensor comprises a plurality of electrodes, wherein the at least one capacitance value is indicative, representative and/or descriptive of a capacitance between at least two electrodes, e.g. at least two directly neighboring or adjoining electrodes, of the plurality of electrodes. For instance, the capacitive sensor may be configured to provide one or more sensor signals, wherein each sensor signal may be indicative of the capacitance between a pair of electrodes of the plurality of electrodes. Further, the control circuitry may be configured to process each sensor signal and determine the capacitance value for each pair of electrodes of the plurality electrodes.

According to an embodiment, the at least two electrodes of the plurality of electrodes are arranged adjacent to each other. Alternatively or additionally, the at least two electrodes are directly neighboring each other.

According to an embodiment, the capacitive sensor comprises a plurality of electrodes, wherein the control circuitry is configured to determine a plurality of capacitance values, each capacitance value being indicative, representative and/or descriptive of a capacitance between a pair of electrodes of the plurality of electrodes. Generally, using a plurality of electrodes and determining the capacitance values of the capacitances between pairs of the electrodes can increase an accuracy and precision of the determination of the position and/or orientation of the user's head relative to the optical system and/or the electrodes. Accordingly, also a precision and accuracy in positioning the optical system may be increased.

According to an embodiment, the capacitive sensor comprises a plurality of electrodes arranged in a three-dimensional configuration. Using a three-dimensional configuration of the electrodes may allow to infer and/or derive precise and/or accurate three-dimensional information, e.g. information in three-dimensional space, from the one or more sensor signals. Accordingly, a positional control and/or a speed control of the optical system in three-dimensional space may be improved.

According to an embodiment, the capacitive sensor comprises a plurality of electrodes arranged in at least one of a semi-spherical configuration, a spherical configuration and an arc-shaped configuration. Such configurations may arc-like bridge over and/or at least partly encompass at least a part of the head of the user, such as e.g. a forehead. This may allow determining the position and/or orientation of the user's head with respect to the capacitive sensor, the optical system and/or the at least part thereof in three-dimensional space with high precision and accuracy.

According to an embodiment, the capacitive sensor comprises an arc-shaped array of electrodes and at least one further arc-shaped array of electrodes, wherein the arc-shaped array and the at least one further arc-shaped array are directed and/or extend in different spatial directions. Also such configuration may allow determining the position and/or orientation of the user's head with respect to the capacitive sensor, the optical system and/or the at least part thereof in three-dimensional space with high precision and accuracy.

According to an embodiment, the control circuitry is configured to determine a plurality of capacitance values, each capacitance value being indicative, representative and/or descriptive of the capacitance in the vicinity of at least one of the electrodes. The control circuitry is further configured to compare each of the plurality of determined capacitance values with at least one reference value, and to actuate the at least one actuator to move the optical system, such that at least one of the orientation of the head of the user and the at least part of the optical system, the relative distance between the user's head and the at least part of the optical system and a relative position of the head of the user and the at least part of the optical system is substantially constant. Alternatively or additionally, the control circuitry is configured to actuate the at least one actuator to move the optical system, such that at least one of a relative velocity of the head of the user and the at least part of the optical system, and a relative acceleration of the head of the user and the at least part of the optical system is minimized. The plurality of capacitance values determined by the control circuitry may for instance refer to capacitance values determined at different times. The control circuitry may be configured to determine, based on the comparison of the capacitance values with the reference capacitance values, the relative orientation, the relative position, the relative velocity and/or the relative acceleration of the user's head with respect to the at least part of the optical system. Further, the control circuitry may actuate the at least one actuator and/or to move, displace and/or position the optical system, such that the relative position, the relative orientation and/or the relative distance between the user's head and the at least part of the optical system is substantially constant. Alternatively or additionally, the control circuitry may be configured to actuate the at least one actuator such that the optical system is moved and/or displaced with a velocity and/or an acceleration which substantially corresponds to and/or matches the velocity and/or acceleration of the head of the user, e.g. thereby minimizing the relative velocity and/or the relative acceleration of the user's head with respect to the at least part of the optical system. In other words, the optical system may be actuated in correspondence with a movement of the user's head. This may allow not only a positional control based on moving the user's head, but also a speed and/or acceleration control. Hence, the speed, velocity and/or acceleration, by which the optical system is moved, can be controlled by the user based on moving its head with a certain velocity and/or acceleration. Generally, this may provide a comprehensive control of the optical system.

According to an embodiment, the control circuitry is configured to determine a sequence of time-related capacitance values. In other words, the control circuitry may be configured to determine the capacitance values and/or the capacitance in the vicinity of the at least one electrode as a function of time. Accordingly, the control circuitry may be configured to monitor the capacitance (values) and/or determine the capacitance (values) over time.

According to an embodiment, the control circuitry is configured to determine, based on the determined sequence of time related capacitance values, at least one of a velocity of the head of the user, an acceleration of the head of the user, a movement of the head of the user, a positional change of the head of the user, a time period during which the head of the user moves, and a time period during which the head of the user is static. Accordingly, the control circuitry may be configured to derive comprehensive information about a movement of the user's head, which information may be used to comprehensively control the optical system and/or the microscope.

According to an embodiment, the control circuitry is configured to actuate the at least one actuator to move the optical system based on comparing the at least one capacitance value with the at least one reference value and based on determining whether and/or if at least one boundary condition is fulfilled. By taking the at least one boundary condition into account, the control of microscope may be further improved for the user.

According to an embodiment, the at least one boundary condition is at least one of:
a minimum deviation between the determined at least one capacitance value and the at least one reference value;
a maximum deviation between the determined at least one capacitance value and the at least one reference value;
a minimum relative velocity between the head of the user and the at least part of the optical system;
a maximum relative velocity between the head of the user and the at least part of the optical system;
a minimum relative acceleration between the head of the user and the at least part of the optical system;
a maximum relative acceleration between the head of the user and the at least part of the optical system;
a minimum time period during which a determined sequence of time-related capacitance values is substantially constant; and
a maximum time period during which a determined sequence of time-related capacitance values is substantially constant.

By way of example, the control circuitry may only move the optical system if the minimum deviation between the determined at least one capacitance value and the at least one reference value, the minimum relative velocity, the minimum relative acceleration and/or the minimum time period is reached and/or exceeded. This may allow to compensate for short-term movements of the user's head which may not be intended by the user to result in a movement and/or displacement of the optical system. Alternatively or additionally, the control circuitry may only move the optical system if the maximum deviation between the determined at least one capacitance value and the at least one reference value, the maximum relative velocity, the maximum relative acceleration and/or the maximum time period is not exceeded and/or reached. This may allow to avoid movements of the optical system, e.g. if the user moves its head by a comparatively large distance, with a comparatively high velocity and/or with a comparatively high acceleration. Such head movements may for instance be performed unintentionally by the user or e.g. when the user moves its head away from the microscope. In general, taking one or more of such boundary conditions into account may prevent the optical system from being moved unintentionally by the user. Also, the overall control of the microscope may be improved.

According to an embodiment, the control circuitry is configured to determine a sequence of time-related capacitance values, wherein the control circuitry is configured to actuate the at least one actuator to move the optical system only if the capacitance values determined over a predetermined period of time are substantially constant and/or are within a predetermined range of capacitance values. This way, the control of the microscope and/or the optical system by means of the user's head may be activated by the user based on keeping its head at a substantially fixed position over the predetermined period of time.

According to an embodiment, the control circuitry is configured to determine a sequence of time-related capacitance values, determine at least one predefined pattern of at least a subset of the determined capacitance values, determine a head movement associated with the at least one predefined pattern, and control at least one function of the robotic microscope in accordance with the determined head movement. Accordingly, the control circuitry may be configured to derive from the plurality of capacitance values a certain head movement based on determining and/or identifying the predefined pattern. In correspondence with the identified pattern and/or the determined head movement, the function of the microscope can be controlled, wherein the function can relate to any function of the microscope. This way, a gesture control for controlling arbitrary functions of the microscope based on moving the head can be employed.

According to an embodiment, the at least one function of the robotic microscope is at least one of activating an augmentation feature of the microscope, deactivating an augmentation feature of the microscope, actuating a zoom of the optical system, capturing at least one image with the microscope, activating a set of reference values for the capacitance, and generating a control signal for controlling a tracking system coupled to the microscope to activate or deactivate the tracking system. Accordingly, an overall control of the microscope based on capacitive sensing and/or based on head movements of the user can be employed.

According to an embodiment, the control circuitry is further configured to process tracking data of a tracking system for tracking a surgical instrument and to activate the capacitive sensor if the surgical instrument is located in a field of view of the optical system. Alternatively or additionally, the control circuitry is configured to process image data of the optical system, determine a state of a surgical procedure and activate the capacitive sensor in dependence of the determined state of the surgical procedure. This may allow to ensure that the optical system is only moved in accordance with a movement of the user's head, e.g. when a surgical procedure is performed and/or when in the determined state of the surgical procedure, the optical system should preferably be moved in accordance with the movement of the user's head. On the other hand, unintentional movements of the optical system, e.g. during a state of the surgical procedure, during which the optical system should not be moved, can be avoided in an effective way.

In a second aspect, the present disclosure relates to the use of a robotic microscope, as described hereinabove and hereinbelow, for a medical and/or surgical procedure.

According to a third aspect of the disclosure, there is provided a method of operating a robotic microscope, as described hereinabove and hereinbelow. The method comprises at least the following steps:
determining, with a control circuitry of the microscope, at least one capacitance value based on processing a sensor signal of a capacitive sensor of the microscope;
comparing the determined at least one capacitance value with at least one reference value; and
actuating, in dependence of and/or in accordance with the comparison of the at least one capacitance value with the at least one reference value, at least one actuator of the microscope, thereby adjusting a position of the optical system in at least one spatial direction, such that a distance between a head of a user of the microscope and at least a part of the optical system and/or an orientation of the head of the user and the at least part of the optical system is substantially constant.

It should be noted that the method according to the first aspect of the present disclosure does not involve or in particular comprise or encompass any invasive step, which would represent a substantial physical interference with the body requiring professional medical expertise to be carried out and entailing a substantial health risk even when carried out with the required professional care and expertise. Particularly, the invention does not involve or in particular comprise or encompass any surgical or therapeutic activity. The invention is instead directed as applicable to the mere control of a robotic microscope. For this reason alone, no surgical or therapeutic activity and in particular no surgical or therapeutic step is necessitated or implied by carrying out the method according to the first aspect.

Further, it should be noted that the method according to the third aspect of the disclosure is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

In a fourth aspect, the present disclosure is directed to a computer program which, when running on at least one processor (for example, a processor) of at least one computer (for example, a computer and/or a computer, controller and/or control circuitry of the robotic microscope) or when loaded into at least one memory (for example, a memory) of at least one computer (for example, a computer), causes the at least one computer to perform the above-described method according to the third aspect. The present disclosure may alternatively or additionally relate to a (physical, for example electrical, for example technically generated) signal wave, for example a digital signal wave, carrying information which represents the program, for example the aforementioned program, which for example comprises code means which are adapted to perform any or all of the steps of the method according to the third aspect. A computer program stored on a disc is a data file, and when the file is read out and transmitted it becomes a data stream for example in the form of a (physical, for example electrical, for example technically generated) signal. The signal can be implemented as the signal wave which is described herein. For example, the signal, for example the signal wave is constituted to be transmitted via a computer network, for example LAN, WLAN, WAN, for example the internet. The invention according to the fourth aspect therefore may alternatively or additionally relate to a data stream representative of the aforementioned program.

In a fifth aspect, the present disclosure is directed to a non-transitory computer-readable program storage medium on which the program according to the fourth aspect is stored.

In a sixth aspect, the present disclosure is directed to at least one computer (for example, a computer), comprising at least one processor (for example, a processor) and at least one memory (for example, a memory), wherein the program according to the fourth aspect is running on the processor or is loaded into the memory, or wherein the at least one computer comprises the computer-readable program storage medium according to the fifth aspect.

According to a seventh aspect, the present disclosure relates to a use of a capacitive sensor for controlling a robotic microscope, as described hereinabove and hereinbelow.

It is emphasized that features, functions, elements and/or steps, which are described above and in the following with reference to one aspect of the invention or disclosure, equally apply to any other aspect of the invention or disclosure described above and in the following. Particularly, features and/or steps, as described above and in the following, with reference to the robotic microscope according to the first aspect, equally apply to the use according to the second aspect, the method according to the third aspect, the computer program according to the fourth aspect, to the computer-readable medium according to the fifth aspect, to the computer according to the sixth aspect, and/or to the use of the capacitive sensor according to the seventh aspect, and vice versa.

These and other aspects of the disclosure will be apparent from and elucidated with reference to the embodiments described hereinafter.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 shows a robotic microscope according to an exemplary embodiment,

FIG. 2 shows top view of a robotic microscope according to an exemplary embodiment;

FIG. 3 shows a side view robotic microscope according to an exemplary embodiment;

FIG. 4 shows a flowchart illustrating steps of a method operating a robotic microscope according to an exemplary embodiment.

The figures are schematic only and not true to scale. In principle, identical or like parts, elements and/or steps are provided with identical or like reference numerals in the figures.

### DESCRIPTION OF EMBODIMENTS

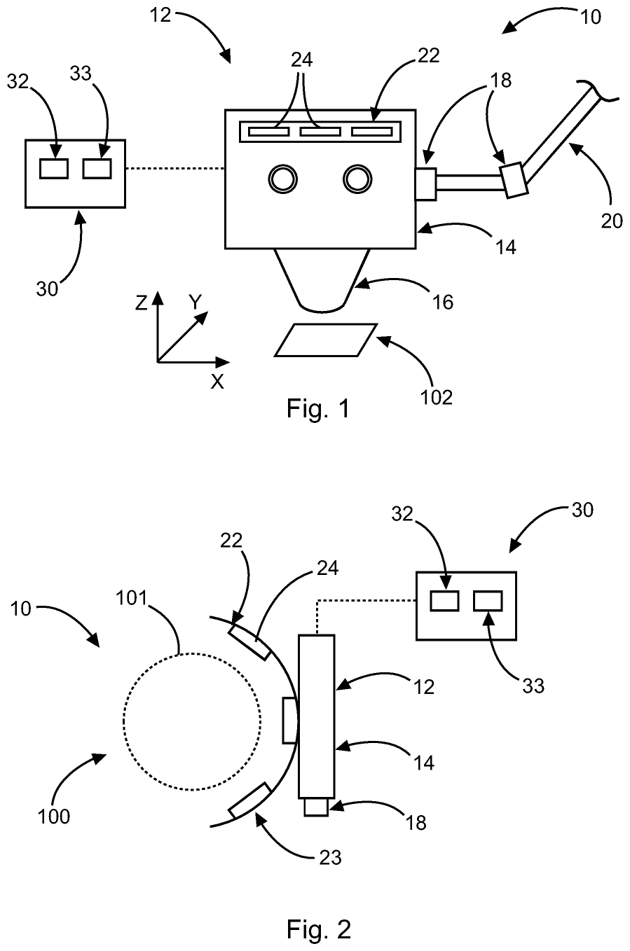

FIG. 1 shows a robotic microscope 10 according to an exemplary embodiment.

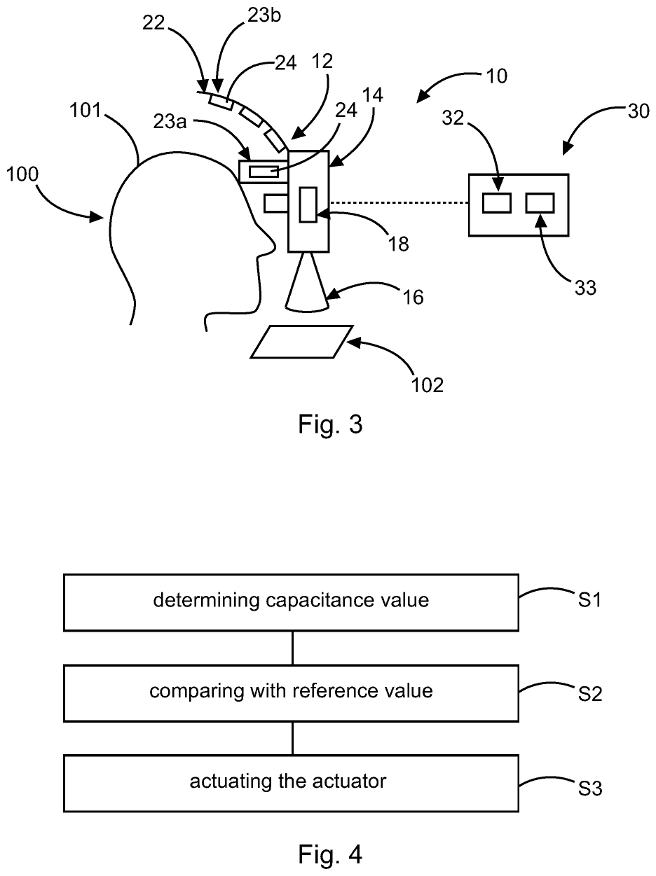

The robotic microscope 10 comprises an optical system 12, which includes an eyepiece 14 and/or ocular 14, through which a user 100 (see FIGS. 2 and 3) may examine and/or view a site of interest 102, such as e.g. a surgical site 102.

For examining and/or viewing the site of interest 102, the optical system 12 further comprises an optics 16, which may e.g. include one or more lenses, one or more mirrors, one or more sensors, e.g. CCD sensors, or the like.

Therein, the optical system 12, the eyepiece 14 and/or the optics 16 may be configured such that the user 100 can view, see and/or examine the site of interest 102 directly based on light emitted or reflected at the site of interest 102 or indirectly based on e.g. a display device arranged in the eyepiece 12.

The microscope 10 further comprises one or more actuators 18 for moving, displacing and/or positioning the optical system 12 in three-dimensional space. In the example of FIG. 1, two actuators 18 are exemplary shown, which are integrated in a support arm 20 or robot arm 20 coupled and/or attached to the optical system 12. The support arm 20 may for instance be mounted on a ceiling or floor, e.g. of an operating theatre, or it may be mounted on a wagon, a stand, or the like. By means of the actuators 18, the optical system 12 can be moved in and/or along various orthogonal spatial directions, such as e.g. the three spatial directions x, y, z as indicated in FIG. 1 by the Cartesian coordinate system shown therein.

Further, the optical system 12 may be rotated by means of the actuators 18 around one or more axes. For instance, each spatial direction x, y, z may define an axis, around which the optical system 12 can be rotated. Apart from that, also a speed and/or velocity, with which the optical system 12 can be moved along the spatial directions and/or rotated around the axes can be controlled and/or adjusted by means of the actuators 18. Accordingly, the microscope 10 of FIG. 1 comprises multiple degrees of freedom, such that the optical system 12 can be freely positioned in three-dimensional space with respect to the site of interest 102, e.g. at a desired viewing angle and/or in a desired viewing direction.

The robotic microscope 10 further comprises a capacitive sensor 22 with at least one electrode 24. In the example shown in FIG. 1, the capacitive sensor 22 comprises in total three electrodes 24, however, any other number of electrodes 24 is conceivable. The capacitive sensor 22 is configured to provide one or more sensor signals indicative of a capacitance in a vicinity of at least one of the electrodes 24. Further, the capacitive sensor 22 and/or the electrodes 24 thereof shown in the example of FIG. 1 are arranged in a substantially flat configuration on the optical system 12 and/or on the eyepiece 14 thereof. It should be noted, however, that other configurations of the optical system 12 are conceivable, e.g. those described with reference to FIGS. 2 and 3.

Further, the capacitive sensor 22 and/or the electrodes 24 thereof are mounted and/or attached to the eyepiece 14, such that the capacitive sensor 22 and/or the electrodes 24 move in accordance and/or correspondence with a movement of the optical system 12 and/or the eyepiece 14.

The microscope 10 further comprises a control circuitry 30 configured to process the one or more sensor signals of the capacitive sensor 22. Further, the control circuitry 30 is configured to determine and/or derive one or more capacitance values for the capacitances in the vicinity of the one or more electrodes 24 based on the one or more sensor signals. For this purpose, the control circuitry 30 may comprise one or more processors 32 and/or e.g. a memory 33 or data storage device 33.

Therein, a capacitance in a vicinity of one of the electrodes 24 and/or the corresponding capacitance value may be indicative, descriptive and/or representative of at least one of a capacitance between said electrode 24 and a ground (or ground potential), between said electrode 24 and an outer surface of the user's 100 head 101 (see FIGS. 2 and 3), between said electrode 24 and a reference electrode of the capacitance sensor 22 (not shown), and between two electrodes 24 of the capacitance sensor 22, such as e.g. two directly neighboring and/or adjoining electrodes 24.

When the user 100 places its head 101 close to, adjacent to, and/or next to the capacitance sensor 22, e.g. in order to operate the microscope 10 and/or use the eyepiece 14, the electrical permittivity of the material, which may then be provided by the user's 100 head 101, may change and/or affect the capacitance in the vicinity of the one or more electrodes 24 and/or the corresponding capacitance values. Accordingly, based on the one or more sensor signals, the control circuitry 30 may determine, detect and/or sense a positional change and/or a movement of the head 101 of the user 102. This information may then be used by the control circuitry 30 to position, move, displace and/or arrange the optical system 12 in three-dimensional space.

For this purpose, the control circuitry 30 is configured to compare the determined one or more capacitance values with one or more reference values for the one or more capacitances in the vicinity of the one or more electrodes 24. The one or more reference values may e.g. be stored in the memory 33 or data storage device 33. Further, based on this comparison, the control circuitry 30 can actuate the one or more actuators 18, such that a relative or absolute distance between the head 101 of the user 102 and the at least part of the optical system 12, such as e.g. the eyepiece 14, is kept substantially constant. Accordingly, the control circuitry 30 may move the optical system 12 in accordance and/or correspondence with a movement of the user's 100 head 101, e.g. such that the optical system 12 and/or the at least part thereof follows the movement of the head 101 in and/or along one or more spatial directions. This may allow the user 100 to touchlessly navigate and/or position the microscope 10 and/or the optical system 12 in three-dimensional space.

Alternatively or additionally, the control circuitry 30 can be configured to actuate, based on the comparison of the capacitance value(s) with the reference value(s), the one or more actuators 18, such that an orientation of the head 101 of the user 102 and the at least part of the optical system 12, such as e.g. the eyepiece 14, is kept substantially constant. Therein, the orientation may refer to an angular position along one or more of the axes, e.g. as defined by the one or more spatial directions along which the optical system 12 can be moved and/or displaced.

For instance, the control circuitry 30 can be configured to determine a deviation of the determined one or more capacitance values and the one or more reference values for the capacitances, wherein the control circuitry 30 can be configured to actuate the one or more actuators 18 to move the optical system 12, such that the deviation of the one or more capacitance values and the one or more reference values is minimized, thereby keeping the distance between the head 101 of the user 100 and the at least part of the optical system 12 (e.g. the eyepiece 14) and/or the orientation of the head 101 of the user 100 and the at least part of the optical system 12 (e.g. the eyepiece 14) substantially constant.

Moreover, the control circuitry 30 can be configured to determine the one or more capacitances and/or the one or more capacitance values over time. In other words, a sequence of time related capacitance values may be determined for the one or more electrodes 24. Based on the determined sequence of time-related capacitance values, the control circuitry 30 can determine, compute and/or calculate at least one of a velocity of the head 101 of the user 100, an acceleration of the head 101 of the user 100, a movement of the head 101 of the user 100, a positional change of the head 101 of the user 100, a time period during which the head 101 of the user 100 moves, and a time period during which the head 101 of the user 100 is static. The control circuitry 30 can then actuate the one or more actuators 18, such that the at least part of the optical system 12 (e.g. the eyepiece 14) is moved with a speed and/or an acceleration corresponding to the speed and/or acceleration of the head 101 of the user 100. This may allow the user 100 to not only control a positional movement, but also the speed and/or acceleration, with which the optical system 12 is moved.

Further, the control circuitry 30 can be configured to actuate the one or more actuators 18 based on determining whether at least one boundary condition is fulfilled. Such boundary condition may, for instance, be a minimum deviation between the determined at least one capacitance value and the at least one reference value, a maximum deviation between the determined at least one capacitance value and the at least one reference value, a minimum relative velocity between the head of the user and the at least part of the optical system, a maximum relative velocity between the head of the user and the at least part of the optical system, a minimum relative acceleration between the head of the user and the at least part of the optical system, a maximum relative acceleration between the head of the user and the at least part of the optical system, a minimum time period during which a determined sequence of time-related capacitance values is substantially constant, and a maximum time period during which a determined sequence of time-related capacitance values is substantially constant. By taking into account one or more of such boundary conditions, a smooth and seamless control of the optical system 12 may be provided for the user 100. Apart from that, unintentional movements of the head 101 may be filtered.

Moreover, the control circuitry 30 can be configured to determine a sequence of time-related capacitance values, and to actuate the one or more actuators 18 to move the optical system 12 only if the capacitance values determined over a predetermined period of time are substantially constant and/or are within a predetermined range of capacitance values. This may allow a user e.g. to wilfully activate the positional control of the microscope 10 by remaining at a static position for the predetermined period of time. Also, unintentional movements and/or changes in the capacitances occurring on a short time scale can be filtered therewith.

Apart from that, the control circuitry 30 can be configured to determine a sequence of time-related capacitance values, and to determine at least one predefined pattern of at least a subset of the determined capacitance values. Further, the control circuitry 30 can be configured to determine a head movement associated with the at least one predefined pattern, and control at least one function of the robotic microscope 10 in accordance with the determined head movement. Such head movement may, for instance, be a nod, a nodding, a shake, a shaking of the head 101 or any other head movement. Further, the function of the microscope may, for example, be at least one of activating an augmentation feature of the microscope 10, deactivating an augmentation feature of the microscope 10, actuating a zoom of the optical system 12, capturing at least one image with the microscope 12, activating a set of reference values for the capacitance, and generating a control signal for controlling a tracking system (not shown) coupled to the microscope 10 to activate or deactivate the tracking system. Accordingly, a comprehensive overall control for controlling any function of the microscope 10 can be employed based on the capacitance sensor 22.

FIG. 2 shows a top view of a robotic microscope 10 according to an exemplary embodiment. If not stated otherwise, the robotic microscope 10 of FIG. 2 comprises the same features, functions and/or elements as the robotic microscope 10 of FIG. 1.

In the example shown in FIG. 2, the capacitive sensor 22 comprises a plurality of electrodes 24 arranged in a three-dimensional configuration. In particular, the electrodes 24 are arranged in an arc-shaped and/or semi-spherical configuration. More specifically, the capacitive sensor 22 comprises an arc-shaped array 23 of electrodes 24. Accordingly, the capacitive sensor 22 and/or the electrodes 24 at least partly encompass the head 101 of the user 100. Generally, this may allow to infer three-dimensional information about a movement and/or displacement of the head 101 with high precision and accuracy.

FIG. 3 shows a side view of a robotic microscope 10 according to an exemplary embodiment. If not stated otherwise, the robotic microscope 10 of FIG. 3 comprises the same features, functions and/or elements as the robotic microscopes 10 of FIGS. 1 and 2.

In the example shown in FIG. 3, the capacitive sensor 22 comprises a first arc-shaped array 23a, which may correspond to the arc-shaped array 23 of the capacitive sensor 22 of FIG. 2. The capacitive sensor 22 comprises a second arc-shaped array 23b, which spans and/or bridges at least partly across or over the head 101 of the user 100. Accordingly, the second arc-shaped array 23b may be arranged above the head 101 of the user 100.

The first arc-shaped array 23a is directed and/or extends along a first direction (or arc-shaped axis) and the second arc-shaped array 23b extends and/or is directed along a second direction (or arc-shaped axis) different than the first direction (or arc-shaped axis). Also this configuration of the capacitive sensor 22 may allow to infer three-dimensional information about a movement and/or displacement of the head 101 with high precision and accuracy.

FIG. 4 shows a flowchart illustrating steps of a method of operating a robotic microscope 10 according to an exemplary embodiment. The microscope 10 may for example be any of the microscopes 10 described with reference to the foregoing figures.

The method comprises a step S1 of determining, with a control circuitry 30 of the microscope 10, at least one capacitance value based on processing one or more sensor signals of a capacitive sensor 22 of the microscope 10.

The method encompasses a further step S2 of comparing the determined at least one capacitance value with at least one reference value.

Further, the method comprises a step S3 of actuating, in dependence of the comparison of the at least one capacitance value with the at least one reference value, at least one actuator 18 of the microscope 10, thereby adjusting a position of the optical system 12 in at least one spatial direction, such that a distance between a head 101 of a user 100 and at least a part of the optical system 12 (e.g. the eyepiece 14) and/or an orientation of the head 101 of the user 100 and the at least part of the optical system 12 (e.g. the eyepiece 14) is substantially constant.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A robotic microscope, the robotic microscope comprising:
an optical system being movable in one or more spatial directions;
at least one actuator configured to move the optical system in the one or more spatial directions;
a capacitive sensor with at least one electrode coupled to at least a part of the optical system, wherein the capacitive sensor is configured to provide a sensor signal indicative of a capacitance in a vicinity of the at least one electrode; and
a control circuitry configured to determine, based on processing the sensor signal, at least one capacitance value for the capacitance;
wherein the control circuitry is further configured to actuate, based on comparing the at least one capacitance value with at least one reference value for the capacitance, the at least one actuator to move the optical system, such that at least one of a distance between a head of a user and the at least part of the optical system and or an orientation of the head of the user and the at least part of the optical system is substantially constant.

2. The robotic microscope according to claim 1, wherein the control circuitry is configured to determine a deviation of the at least one capacitance value and the at least one reference value; and wherein the control circuitry is configured to actuate the at least one actuator to move the optical system, such that the deviation of the at least one capacitance value and the at least one reference value is minimized, thereby keeping at least one of the distance between the head of the user and the at least part of the optical system or the orientation of the head of the user and the at least part of the optical system substantially constant.

3. The robotic microscope according to claim 1, wherein the at least one capacitance value is indicative of a capacitance between at least one of the at least one electrode and the head of the user or the at least one electrode and ground.

4. The robotic microscope according to claim 1, wherein the capacitive sensor comprises a plurality of electrodes; and wherein the at least one capacitance value is indicative of a capacitance between at least two electrodes of the plurality of electrodes.

5. The robotic microscope according to claim 4, wherein the at least two electrodes are at least one of arranged adjacent to each other or w arranged directly neighboring each other.

6. The robotic microscope according to claim 1, wherein the capacitive sensor comprises a plurality of electrodes; and wherein the control circuitry is configured to determine a plurality of capacitance values, each capacitance value being indicative of a capacitance between a pair of electrodes of the plurality of electrodes.

7. The robotic microscope according to claim 1, wherein the capacitive sensor comprises a plurality of electrodes arranged in a three-dimensional configuration.

8. The robotic microscope according to claim 1, wherein the capacitive sensor comprises a plurality of electrodes arranged in at least one of a semi-spherical configuration, a spherical configuration and an arc-shaped configuration.

9. The robotic microscope according to claim 1, wherein the capacitive sensor comprises an arc-shaped array of electrodes and at least one further arc-shaped array of electrodes; and wherein the arc-shaped array and the at least one further arc-shaped array are at least one of directed in different directions or extend in different directions.

10. The robotic microscope according to claim 7, wherein the control circuitry is configured to:
determine a plurality of capacitance values, each capacitance value being indicative of the capacitance in the vicinity of at least one of the electrodes;
compare each of the plurality of determined capacitance values with at least one reference value; and
actuate the at least one actuator to move the optical system, such that at least one of the orientation of the head of the user and the at least part of the optical system and a relative position of the head of the user and the at least part of the optical system is substantially constant, or such that at least one of a relative velocity of the head of the user and the at least part of the optical system and a relative acceleration of the head of the user and the at least part of the optical system is minimized.

11. The robotic microscope according to claim 1, wherein the control circuitry configured to determine a sequence of time-related capacitance values.

12. The robotic microscope according to claim 11, wherein the control circuitry is configured to determine, based on the determined sequence of time related capacitance values, at least one of a velocity of the head of the user, an acceleration of the head of the user, a movement of the head of the user, a positional change of the head of the user, a time period during which the head of the user moves, and a time period during which the head of the user is static.

13. The robotic microscope according to claim 1, wherein the control circuitry is configured to actuate the at least one actuator to move the optical system based on comparing the at least one capacitance value with the at least one reference value and based on determining whether at least one boundary condition is fulfilled.

14. The robotic microscope according to claim 13, wherein the at least one boundary condition is at least one of:
a minimum deviation between the determined at least one capacitance value and the at least one reference value;
a maximum deviation between the determined at least one capacitance value and the at least one reference value;
a minimum relative velocity between the head of the user and the at least part of the optical system;
a maximum relative velocity between the head of the user and the at least part of the optical system;
a minimum relative acceleration between the head of the user and the at least part of the optical system;
a maximum relative acceleration between the head of the user and the at least part of the optical system;
a minimum time period during which a determined sequence of time-related capacitance values is substantially constant; and
a maximum time period during which a determined sequence of time-related capacitance values is substantially constant.

15. The robotic microscope according to claim 1, wherein the control circuitry is configured to determine a sequence of time-related capacitance values; and wherein the control circuitry is configured to actuate the at least one actuator to move the optical system only if the time-related capacitance values determined over a predetermined period of time are at least one of substantially constant or are within a predetermined range of capacitance values.

16. The robotic microscope according to claim 1, wherein the control circuitry is configured to:
determine a sequence of time-related capacitance values;
determine at least one predefined pattern of at least a subset of the determined capacitance values;
determine a head movement associated with the at least one predefined pattern; and
control at least one function of the robotic microscope in accordance with the determined head movement.

17. The robotic microscope according to claim 16, wherein the at least one function of the robotic microscope is at least one of activating an augmentation feature of the robotic microscope, deactivating an augmentation feature of the robotic microscope, actuating a zoom of the optical system, capturing at least one image with the robotic microscope, activating a set of reference values for the capacitance, and generating a control signal for controlling a tracking system coupled to the robotic microscope to activate or deactivate the tracking system.

18. The robotic microscope according to claim 1, wherein the control circuitry is further configured to process tracking data of a tracking system for tracking a surgical instrument and to activate the capacitive sensor if the surgical instrument is located in a field of view of the optical system.

19. (canceled)

20. A method of operating a robotic microscope, the method comprising:
determining, with a control circuitry of the robotic microscope, at least one capacitance value based on processing a sensor signal of a capacitive sensor of the robotic microscope;
comparing the determined at least one capacitance value with at least one reference value; and
actuating, in dependence of the comparison of the at least one capacitance value with the at least one reference value, at least one actuator of the robotic microscope, thereby adjusting a position of the optical system in at least one spatial direction, such that at least one of a distance between a head of a user and at least a part of the optical system or an orientation of the head of the user and the at least part of the optical system is substantially constant.

21. The robotic microscope according to claim 1, wherein the control circuitry is configured to process image data of the optical system, determine a state of a surgical procedure and activate the capacitive sensor in dependence of the determined state of the surgical procedure.
